(19) United States
(12) Patent Application Publication
(10) Pub. No.: US 2008/0199448 A1
(43) Pub. Date: Aug. 21, 2008

(54) ENZYME COMPOSITION FOR IMPROVING FOOD DIGESTION

(76) Inventors: Mairi R. Ross, Jerome, AZ (US); Yoon Mo Kang, Scottsdale, AZ (US)

Correspondence Address: JAECKLE FLEISCHMANN & MUGEL, LLP, 190 Linden Oaks, ROCHESTER, NY, 14625-2812, US

(21) Appl. No.: 12/032,065

(22) Filed: Feb. 15, 2008

Related U.S. Application Data
(60) Provisional application No. 60/890,282, filed on Feb. 16, 2007.

(51) Int. Cl. A61K 9/48; A61K 38/54

(52) U.S. Cl. 424/94.2; 424/463

(57) ABSTRACT

An orally administered composition for improving food absorption and digestion contains therapeutically effective dosages of digestive enzymes and L-glutamine as active ingredients. The digestive enzymes include at least one each of a lipase, a protease, and an amylase, and at least a portion of each of these enzymes is enteric coated.

### CROSS-REFERENCE TO RELATED APPLICATIONS

This application claims the benefit of U.S. Provisional Patent Application Ser. No. 60/890,282, filed Feb. 16, 2007.

### FIELD OF THE INVENTION

The present invention relates to enzyme compositions and, more particularly to orally administered enzyme compositions for improving the digestion and absorption of food.

### BACKGROUND OF THE INVENTION

The normal digestive process entails the breakdown of food into small particles by enzymes in the stomach and small intestine. Some individuals do not have sufficient enzymes for normal food digestion; as a result, undigested food remains in the stomach and may result in an excess amount of stomach acid. If food is not effectively digested in the stomach, it may pass into the small intestine in a form that is not absorbable, resulting in malabsorption and nutritional deficiency, which may also lead to increased gas, bloating and slower food transit.

Foods, especially carbohydrates and fats, are also digested by specific enzymes in the small intestine. If the small intestine has an insufficient amount of the appropriate enzymes for efficient digestion, nutrients are not absorbed, and the undigested food moves to the colon where it undergoes excess bacterial fermentation, resulting in excess gas and bloating. Undigested fats either in the stomach or small intestine may reflux and weaken the lower esophageal sphincter, allowing acid into the esophagus.

Foods are broken down both in the stomach and in the small intestine. Therefore digestive enzymes must operate in the high-acid, low pH conditions of the stomach, where almost all protein and simple carbohydrate digestion occurs, and in the less acidic environment of the small intestine, where almost all complex carbohydrates and fats are digested.

Sipos, U.S. Pat. No. 4,079,125, the disclosure of which is incorporated herein by reference, describes an enteric-coated digestive enzyme-containing composition that comprises a concentrate of enzymes from the group consisting of pancreatic, plant-derived, and microbial-derived enzymes, together with a binder and a disintegrant.

Bilton, U.S. Pat. No. 4,447,412, the disclosure of which is incorporated herein by reference, describes an enzyme-containing digestive aid composition that contains a mixture of enteric coated beads incorporating pancreatic and proteolytic enzymes, in combination with granules comprising a chloretic agent, a hydrochloric acid salt, and pepsin.

Handel et al., U.S. Pat. No. 5,387,422, the disclosure of which is incorporated herein by reference, describes a food supplement composition that comprises at least one acid protease fungal enzyme and at least one semi-alkaline protease fungal enzyme.

Margolin et al., U.S. 2001/0046493, the disclosure of which is incorporated herein by reference, describes a composition comprising a crosslinked non-fungal lipase crystal that is resistant to proteolysis, low pH, and elevated temperature, together with a protease and an amylase, wherein the lipase crystal is active at a pH range of about 2.0 to 9.0.

Galle et al., U.S. 2004/0057944, the disclosure of which is incorporated herein by reference, describes an enzyme mixture useful to treat digestive disorders that comprises: a concentrated lipase of Rhizopus delemar, a neutral protease of Aspergillus melleus, and an amylase of Aspergillus oryzae. One or more of the enzymes may be coated with an enteric layer.

Margolin et al., U.S. 2006/012017, the disclosure of which is incorporated herein by reference, describes a composition for treating pancreatic insufficiency that comprises lipase, protease, and amylase as active ingredients, wherein the ratio of lipase, protease, and amylase is about 1:1:0.15 USP units. The reference further teaches that the composition may include as inactive ingredients a wide variety of inorganic and organic excipients, including cellulose, silicon dioxide, magnesium stearate, talc, glycols, amino acids, carbohydrates such as mono-, di- and poly-saccharides, and inorganic chloride and phosphate salts.

The amino acid L-glutamine supports the normal regeneration of the lining of the small intestine and helps to prevent damage to the intestine caused by high levels of protein enzymes. A healthy intestinal lining helps promote the absorption of food nutrients by the small intestine. The importance of L-glutamine to the digestive system in maintaining healthy intestinal mucosa is discussed by Pirisi, “Glutamine:The Conditionally Essential Amino Acid” in LifeExtension LE Magazine, August 2003.

### SUMMARY OF THE INVENTION

The present invention is directed to an orally administered composition for improving food absorption and digestion that comprises therapeutically effective dosages of digestive enzymes and L-glutamine and, optionally, an oligosaccharide as active ingredients. The digestive enzymes include, but are not limited to, at least one each of a lipase, a protease, and an amylase, and at least a portion of each of these enzymes is enteric coated.

### DETAILED DESCRIPTION OF THE INVENTION

In accordance with the present invention, an orally administered composition for improving the absorption and digestion of food comprises therapeutically effective dosages of active ingredients comprising the amino acid L-glutamine and a combination of digestive enzymes that breaks down proteins, fats, carbohydrates, fibers in the stomach and small intestine, enabling the nutrients of the food to be more completely absorbed in the small intestine. At least a portion of each of the lipase, protease, and amylase enzymes included in the composition is enteric coated.

Pancreatin is a substance containing enzymes, principally amylase, lipase, and protease, obtained from the pancreas of a hog or an ox. A milligram of pancreatin contains not less than 25 USP Units of amylase activity, not less than 2.0 USP Units of lipase activity, and not less than 25 USP Units of protease activity. Pancreatin of a higher digestive power may be labeled as a whole-number multiple of the three minimum activities or may be diluted by admixture with lactose, or with sucrose containing not more than 3.25 percent of starch, or with pancreatin of lower digestive power.

One USP Unit of amylase activity is contained in the amount of pancreatin that decomposes starch at an initial rate such that 0.16 μEq of glycosidic linkage is hydrolyzed per minute under the conditions of the assay for amylase activity.

One USP Unit of lipase activity is contained in the amount of pancreatin that liberates 1.0 μLEq of acid per minute at a pH of 90 and 37° under the conditions of the assay for lipase activity.

One USP Unit of protease activity is contained in the amount of pancreatin that under the conditions of the assay for protease activity hydrolyzes casein at an initial rate such that there is liberated per minute an amount of peptides not precipitated by trichloroacetic acid that gives the same absorbance at 280 nm as 15 nmol of tyrosine.

The assays for amylase, lipase, and protease activity are described in detail in USP 24.

In the composition of the present invention, the activities of the included lipase, protease, and amylase are in the ratio of about 0:1:3 USP units, as determined by the procedures just discussed. By contrast, the activity ratio of lipase, protease, and amylase disclosed in the previously Margolin et al., U.S. 2006/012017, is 1:1:0.15 USP units.

The preparation of enteric-coated enzymes is described in the previously mentioned patent, Sipos, U.S. Pat. No. 4,079,125. The efficacy of enteric coating of enzymes may be evaluated by soaking the enteric coated enzymes in an acid bath at a pH of 3.0, which approximates the pH of the stomach, for two hours, then placing in a neutral pH 7.0 medium, which approximates the pH of the small intestine, where the enteric coating dissolves and allows the enzymes to be activated. The following results were obtained for the enteric coated protease, lipase, and amylase:

Protease 7.0: >896 PC (FCC)

Lipase: >64 LU (FCC)

Amylase 6.6: >160 BAU (FCC)

All the digestive enzymes included in the composition are vegetarian, i.e., they are derived from plant, fungal, or bacterial sources, as opposed to animal sources. The inclusion of L-glutamine in the composition helps support cell regeneration in the lining of the small intestine and to protect the lining of the small intestine from damage caused by high levels of protein enzymes. Supporting the health of the intestinal lining increases the ability of the intestine to absorb nutrients. A therapeutically effective dosage of glutamine preferably comprises about 50 mg to about 500 mg more preferably, about 100 mg to about 200 mg.

The composition of the invention also preferably includes a therapeutically effective dosage of an oligosaccharide such as,for example, fructooligosaccharide (FOS), a non-digestible carbohydrate that promotes the growth of beneficial bacteria in the colon and supports healthy colon function. FOS works synergistically with the beneficial bacteria in the colon to promote transit regularity, which helps prevent the blockage of food in the stomach and small intestine, thereby producing normal stools. A healthy colon may also prevent the reflux of negative bacteria and toxins back into the small intestine, stomach and esophagus, where they might contribute to intestinal wall and esophageal sphincter damage.

In the composition of the present invention, the protease enzymes preferably include bromelain, papain, or a combination thereof. Other enzymes included in the composition are preferably selected from the group consisting of: β-glucanase, cellulase, glucoamylase, hemicellulase, invertase, lactase, malt diastase, phytase, and combinations thereof.

Preferably, the composition comprises a solid form such as, for example, a powder, a tablet, a capsule, a caplet, a sachet, or an encapsulated liquid. Particularly preferably, the composition is in the form of a capsule.

The composition of the present invention preferably further comprises at least one excipient as an inactive ingredient, the excipient being selected from the group consisting of a filler, a flow agent, a colorant, a flavoring, a dissolving agent, and combinations thereof. Preferably, the excipient, which may comprise up to about 95 weight percent of the composition, is selected from the group consisting of rice maltodextrin, magnesium stearate, and combinations thereof.

In accordance with the present invention, a preferred composition is in the form of a capsule containing about 150 mg of glutamine, about 400 mg of a fructooligosaccharide (FOS), a commercially available example of which is Nutra Flora™ scFOS™, and a total of about 150 mg of the following combination of enzymes:

| | Classi- | | | |
|---|---|---|---|---|
| Enzyme | fication | Origin | Activity (Units*) | |
| Protease 6.0 | Protease | Aspergillus spp. | 9,500 | FCC¹ HUT² |
| Protease 4.5 | Protease | Aspergillus spp. | 18,000 | FCC HUT |
| Protease 3.0 | Protease | Aspergillus spp. | 92.4 | FCC SAPU³ |
| Amylase | Amylase | Aspergillus spp. | 3,000 | FCC DU⁴ |
| Glucoamylase | Amylase | Aspergillus spp. | 4.5 | FCC AGU⁵ |
| Hemicellulase | Other | Aspergillus spp | 320 | FCC HCU⁶ |
| β-glucanase | Other | Aspergillus spp | 5 | FCC BGU⁷ |
| Phytase | Other | Aspergillus spp | 21 | FCC FTU⁸ |
| Lipase | Lipase | Rhizopus spp. | 80 | FCC LU⁹ |
| Cellulase | Other | Aspergillus spp | 90 | FCC CU¹⁰ |
| Lactase | Other | Aspergillus spp | 100 | FCC ALU¹¹ |
| Invertase | Other | Saccharomyces | 110 | FCC SU¹² |
| Malt Diastase | Amylase | Hordeum vulgare | 225,000 | FCC DP¹³ |
| Bromelain | Protease | Ananas comosus | 250,000 | FCC PU¹⁴ |
| Papain | Protease | Carica papaya | 225,000 | FCC PU |
| Protease 7.0 | Protease | Bacillus spp. | 896 | FCC PC¹⁵ |
| (Enteric) | | | | |
| Lipase | Lipase | Rhizopus spp | 64 | FCC LU |
| (Enteric) | | | | |
| Amylase 6.6 | Amylase | Bacillus spp. | 160 | FCC BAU¹⁶ |
| (Enteric) | | | | |

*¹FCC: Food Chemicals Codex, ²HUT: Hemoglobin Units on the Tyrosine basis, ³SAPU: Spectrophotometric Acid Protease Unit, ⁴DU: Dextrinizing Unit, ⁵AGU: AmyloGlucosidase Unit, ⁶HCU: HemiCellulase Unit, ⁷BGU: Beta-Glucanase Unit, ⁸FTU: phytase (FyTase) Unit, ⁹LU: Lipase Unit, ¹⁰CU: Cellulase Unit, ¹¹ALU: Acid Lactase Unit, ¹²SU: Sucrase Unit (INVU: INVtase Unit), ¹³DP: Diastatic Power, ¹⁴PU: Papain Unit, ¹⁵PC: Protease Casein unit, ¹⁶BAU: Bacterial Amylase Unit

All of the enzymes listed in the above table are derived from micro-organic sources, with the exception of malt diastase, bromelain, and papain, which are derived from botanic sources.

In addition to L-glutamine, fructooligosaccharide (FOS), and the enzymes listed above, the preferred composition includes the excipients rice maltodextrin and magnesium stearate.

The enzyme composition shown in the preceding table may be prepared using a multi-step procedure, as follows:

(a) a “gastric formula” is prepared by mixing Aspergillus-fermented and Rhizopus-fermented concentrates with lactase, invertase, bromelain, papain, and maltodextrin with liquid malt extracts in a fluidized bed granulator

(b) an “enteric formula” is prepared by mixing Bacillus-fermented and Rhizopus-fermented concentrates and applying a coating formulation containing hydroxypropyl phthalate and methylcellulose using a fluidized bed coater

(c) the gastric and enteric formulas are mixed prior to encapsulation.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed:

1. An orally administered composition for improving the absorption and digestion of food, said composition comprising therapeutically effective dosages of digestive enzymes and L-glutamine as active ingredients;
wherein said digestive enzymes include at least one each of a lipase, a protease, and an amylase, and at least a portion of each of said lipase, protease, and amylase is enteric coated.

2. The composition of claim 1 wherein said composition improves the digestion of food in the stomach and in the small intestine.

3. The composition of claim 1 further comprising a therapeutical dosage of an oligosaccharide as an active ingredient.

4. The composition of claim 1 wherein said protease includes bromelain, papain, or a combination thereof.

5. The composition of claim 1 further comprising digestive enzymes selected from the group consisting of: β-glucanase, cellulase, glucoamylase, hemicellulase, invertase, lactase, malt diastase, phytase, and combinations thereof.

6. The composition of claim 1 comprising a solid form.

7. The composition of claim 6 wherein said solid form comprises a powder, a tablet, a capsule, a caplet, a sachet, or an encapsulated liquid.

8. The composition of claim 7 wherein said solid form comprises a capsule.

9. The composition of claim 1 further comprising at least one excipient as an inactive ingredient.

10. The composition of claim 9 wherein said excipient is selected from the group consisting of a filler, a flow agent, a colorant, a flavoring, a dissolving agent, and combinations thereof.

11. The composition of claim 9 wherein said excipient comprises up to about 95 weight percent of said composition.

12. The composition of claim 9 wherein said excipient is selected from among rice maltodextrin, magnesium stearate, and combinations thereof.

13. The composition of claim 1 comprising a capsule containing:

| | Classi- | | | |
|---|---|---|---|---|
| Enzyme | fication | Origin | Activity (Units*) | |
| Protease 6.0 | Protease | Aspergillus spp. | 9,500 | FCC HUT |
| Protease 4.5 | Protease | Aspergillus spp. | 18,000 | FCC HUT |
| Protease 3.0 | Protease | Aspergillus spp. | 92.4 | FCC SAPU |
| Amylase | Amylase | Aspergillus spp. | 3,000 | FCC DU |
| Glucoamylase | Amylase | Aspergillus spp. | 4.5 | FCC AGU |
| Hemicellulase | Other | Aspergillus spp | 320 | FCC HCU |
| β-glucanase | Other | Aspergillus spp | 5 | FCC BGU |
| Phytase | Other | Aspergillus spp | 21 | FCC FTU |
| Lipase | Lipase | Rhizopus spp. | 80 | FCC LU |
| Cellulase | Other | Aspergillus spp | 90 | FCC CU |
| Lactase | Other | Aspergillus spp | 100 | FCC ALU |
| Invertase | Other | Saccharomyces | 110 | FCC SU |
| Malt Diastase | Amylase | Hordeum vulgare | 225,000 | FCC DP |
| Bromelain | Protease | Ananas comosus | 250,000 | FCC PU |
| Papain | Protease | Carica papaya | 225,000 | FCC PU |
| Protease 7.0 | Protease | Bacillus spp. | 896 | FCC PC |
| (Enteric) | | | | |
| Lipase (Enteric) | Lipase | Rhizopus spp | 64 | FCC LU |
| Amylase 6.6 | Amylase | Bacillus spp. | 160 | FCC BAU |
| (Enteric) | | | | |

14. The composition of claim 13 wherein said capsule further contains a fructooligosaccharide (FOS).

15. The composition of claim 1 wherein said therapeutically effective dosage of glutamine comprises about 50 mg to about 500 mg.

16. The composition of claim 15 wherein said therapeutically effective dosage of glutamine comprises about 100 mg to about 200 mg.

17. The composition of claim 1 wherein at least a portion of each of said lipase, protease, and amylase is enteric coated using a formulation comprising hydroxypropyl phthalate and methylcellulose.
